(19) United States
(12) Patent Application Publication
(10) Pub. No.: US 2009/0117636 A1
(43) Pub. Date: May 7, 2009

(54) METHOD AND APPARATUS FOR ACCELERATING GROWTH OF E. COLI BY APPLYING ELECTRIC FIELD

(75) Inventors: MinSun KIM, Seoul (KR); Jeongje PARK, Hwaseong-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/028,163

(22) Filed: Feb. 8, 2008

(30) Foreign Application Priority Data
Nov. 5, 2007 (KR) 10-2007-0111993

(51) Int. Cl. C12N 13/00; C12M 1/42

(52) U.S. Cl. 435/173.8; 435/289.1

(57) ABSTRACT

Disclosed are a method and an apparatus for accelerating the growth of E. coli by applying an electric field with a frequency in the range from 1 kHz to 1 MHz. The present invention is applicable to the enhancement of production of heterologous recombinant proteins and metabolites produced by E. coli in such fields as new drug development or research.

### CROSS-REFERENCE TO RELATED APPLICATIONS

This application claims all benefits of Korean Patent Application No. 10-2007-0111993 filed on Nov. 5, 2007 in the Korean Intellectual Property Office, the disclosures of which are incorporated herein by reference.

### BACKGROUND OF THE INVENTION

1. Field of the Invention

The present invention relates to a method and an apparatus for accelerating the growth of E. coli by applying a non-contact A.C. electric field of 1 V to 10 MV/m with a frequency in the range from 1 kHz to 1 MHz or a D.C. electric field of comparable magnitude. The present invention is applicable to the enhancement of production of heterologous recombinant proteins and metabolites produced by E. coli.

2. Description of the Related Art

By transfecting or transforming recombinant genes into eukaryotic or prokaryotic host cells, it is possible to produce heterologous recombinant proteins in large quantity. The heterologous recombinant proteins may be ones for new drug development or research.

To this end, yeast, plant cells or animal cells are mainly used as eukaryotes, and E. coli is mainly used as prokaryotes. Except for special cases, for example, when producing proteins which are toxic to E. coli or when producing human monoclonal antibodies, E. coli is used the most frequently in the production of heterologous proteins. At 37° C., the population of E. coli grows two-fold in only 20 minutes. Such a high growth rate reduces growth time and enables production using inexpensive culture medium, thereby offering advantages in cost and production yield. Also, much is known about the E. coli genome and various cloning vectors are available. Actually, human insulin is produced in large quantity by E. coli using the DNA recombination technology.

Transfection and transformation techniques of eukaryotic and prokaryotic cells are well known in the related art. Examples include heat shock-mediated induction for transfecting prokaryotic cells, bacterial protoplast fusion with intact cells, microinjection and electroporation. Examples of cloning vectors include plasmid, for example, pBR322, pUC series, pBluescript, pACYC177 or pACYC184. Examples of exogenous promoters include E. coli lac, E. coli trp, E. coli phoA and E. coli tac promoters.

The final object of heterologous recombinant protein production is to produce full-length, biologically active proteins with high concentration. However, there is a problem that many of the produced proteins exist as insoluble and inactive inclusion bodies in the cytoplasm. To solve this problem, E. coli is grown at low temperature.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present inventors have found that the growth of E. coli can be accelerated even at low temperature below 37° C. by applying an A.C. electric field of 1 V to 10 MV/m with a frequency in the range from 1 kHz to 1 MHz or a D.C. electric field of comparable magnitude.

In one aspect, the present invention provides a method for accelerating the growth of E. coli comprising applying a non-contact A.C. electric field of 1 V to 10 MV/m with a frequency in the range from 1 kHz to 1 MHz or a D.C. electric field of comparable magnitude, in order to enhance the production of heterologous recombinant proteins and metabolites produced by E. coli.

In another aspect, the present invention provides an apparatus for accelerating the growth of E. coli comprising a container made of an insulating material, two electrodes equipped at the bottom of the container and the outer surface of a lid thereof, and an electric field applying device connected to the two electrodes, wherein the electric field applying device applies an A.C. electric field of 1 V to 10 MV/m with a frequency in the range from 1 kHz to 1 MHz or a D.C. electric field of comparable magnitude.

The above features and advantages of the present invention will be apparent from or are set forth in more detail in the accompanying drawings, which are incorporated in and form a part of this specification, and the following Detailed Description section, which together serve to explain by way of examples of the principles of the present invention.

### BRIEF DESCRIPTION OF THE DRAWINGS

The above and other features of the present invention will now be described in detail with reference to certain exemplary embodiments thereof illustrated in the accompanying drawings which are given hereinbelow by way of illustration only, and thus are not limitative of the present invention, and wherein:

FIG. 1 schematically illustrates an embodiment of the apparatus for accelerating the growth of E. coli according to the present invention;

FIG. 2 shows the graphs comparing OD (optical density) values and ATP levels before and after accelerating the growth of E. coli in accordance with an embodiment of the present invention;

FIG. 3 is the photograph of the formed colony 5 days after the application of an electric field to E. coli; and

FIG. 4 shows the graphs comparing the growth of E. coli by OD values after applying an electric field of frequencies 10 Hz, 100 Hz, 1 kHz, 10 kHz, 100 kHz and 1 MHz.

### DESCRIPTION OF REFERENCE MARKS FOR THE MAIN PORTIONS OF THE DRAWINGS

1: container
2: electrode
3: electric field applying device

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

### DETAILED DESCRIPTION OF THE PREFERRED EMBODIMENTS

Reference will now be made in detail to the preferred embodiments of the present invention, examples of which are illustrated in the drawings attached hereinafter. The embodiments are described below so as to explain the present invention and do not limit the present invention.

In the method for accelerating the growth of E. coli according to the present invention, an A.C. electric field of 1 V to 10 MV/m with a frequency in the range from 1 kHz to 1 MHz or a D.C. electric field of comparable magnitude is applied to accelerate the growth of E. coli, which is used to produce recombinant proteins for new drug development or research. The method of the present invention can be applied to the enhancement of production of heterologous recombinant proteins and metabolites produced by E. coli.

The principle of accelerating the growth of E. coli according to the present invention by applying an electric field is as follows. All cells of living organisms including E. coli require energy to synthesize materials and replicate DNAs, which are needed for the growth and division of cells. This energy is produced and stored in the cell in the form of ATP. When the ATP level is below a critical value, the sustaining of life becomes difficult. ATP is produced in a cell by either of the following two mechanisms. First, glucose or other nutrients are oxidized by cellular respiration to give ATPs. In the other mechanism, protons (H+) are accumulated outside the cellular membrane. When the proton gradient across the cellular membrane reaches a critical level. ADPs are transformed into ATPs as protons are passed through the ATPase. The application of an electric field accelerates the passage of protons or other charged particles. Increased production of ATP resulting therefrom leads to accelerated growth of cells provided that there are sufficient nutrients.

In accordance with an embodiment of the present invention, an A.C. electric field of 1 V to 10 MV/m with a frequency in the range from 1 kHz to 1 MHz or a D.C. electric field of comparable magnitude is applied. When the frequency of the applied A.C. electric field is outside the said range, the intended effect may not be attained, or the growth of E. coli may be inhibited. And, when the magnitude of the applied electric field is smaller than 10 V/m, the effect of accelerating the growth of E. coli may be slight or non-existent. When the magnitude is larger than 1 MV/m, application to production may be difficult because of safety problem.

When an A.C. electric field is applied, the waveform is not particularly limited. A sine wave, a square wave or any arbitrary waveform may be used.

And, in the apparatus for accelerating the growth of E. coli according to an embodiment of the present invention, the electrode may be a metal electrode, a transparent electrode, or a conductive plastic electrode. One of two electrodes is grounded and the other is connected to the electric field applying device.

In an embodiment of the apparatus for accelerating the growth of E. coli according to the present invention, the container may contain a mixture of E. coli and a culture medium. Proteins produced by E. coli include those excreted out of the cell and present in the culture medium, in addition to those dissolved in the cytoplasm or periplasm of E. coli cells. The proteins and metabolites present in the culture medium may be recovered and isolated from the culture medium.

In an embodiment of the present invention, the application of an electric field is performed by the non-contact method, that is, without contacting the electrode with E. coli or a solution containing E. coli. FIG. 1 schematically illustrates an embodiment of the apparatus for accelerating the growth of E. coli according to the present invention, wherein the electric field is applied by the non-contact method. To this end, a container 1 made of an insulating material holding E. coli or a solution containing E. coli is disposed between the two electrodes 2. One of the two electrodes 2 is equipped at the bottom of the container 1 and the other at the outer surface of the lid of the container 1. Then, one of the electrode is connected to the electric field applying device 3, and then an electric field is applied by the non-contact method.

### EXAMPLES

The following examples illustrate the present invention and are not intended to limit the same.

### Example 1

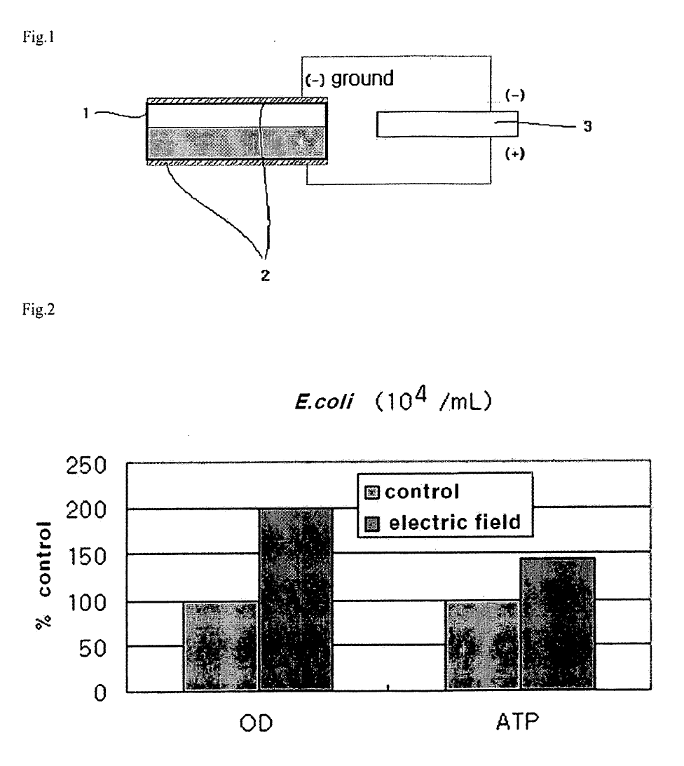

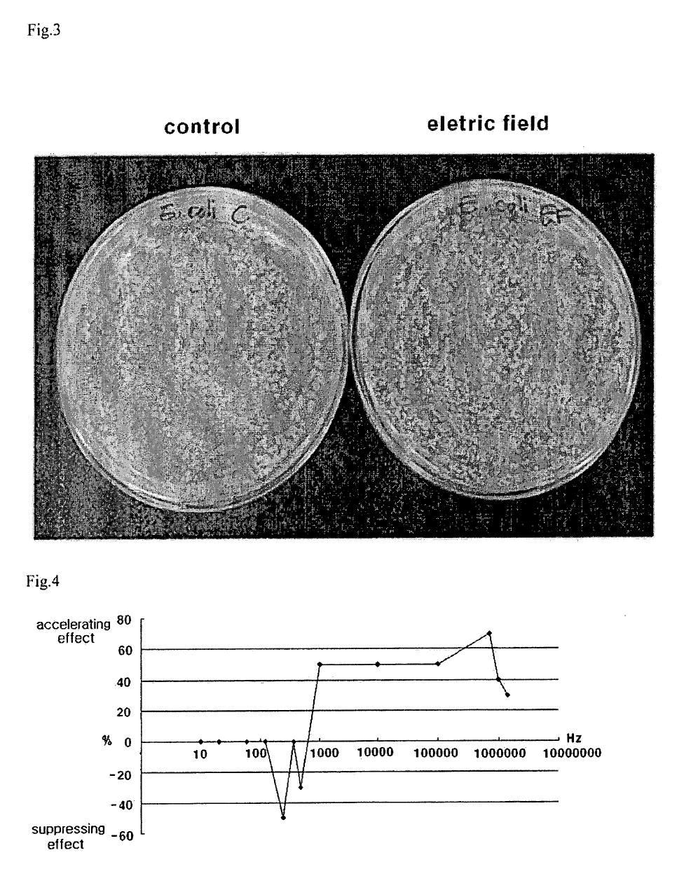

E. coli was cultured in an E. coli culture medium to a concentration of 10⁴ CFU/mL. The mixture of E. coli and the culture medium was placed on a 96-well plate made of insulating polypropylene. At 10° C., an A.C. sine electric field of 700 kHz was applied with a magnitude of 100 V/cm by the non-contact method, as illustrated in FIG. 1. Five days after the application of the electric field, cell growth was evaluated from OD (optical density) value of the mixture of E. coli and the culture medium at 600 nm. ATP level, and cell counts. FIG. 2 shows the graphs comparing OD values and ATP levels before and after accelerating the growth of E. coli in accordance with an embodiment of the present invention, and FIG. 3 is the photograph of the formed colony 5 days after the application of the electric field.

As can be seen from FIG. 2 and FIG. 3, the concentration of E. coli and the ATP level were high when an electric field of 700 kHz was applied, which confirms that the growth of E. coli was accelerated.

### Example 2

E. coli was cultured in an LB culture medium. The mixture of E. coli and the culture medium was placed on a 96-well plate made of insulating polypropylene. At 10° C., A.C. sine electric fields of 10 Hz, 100 Hz, 1 kHz, 10 kHz, 100 kHz and 1 MHz were applied with a magnitude of 100 V/cm by the non-contact method, as illustrated in FIG. 1. Cell growth was evaluated from OD values of the mixture of E. coli and the culture medium at 600 nm. As can be seen from FIG. 4, the growth of E. coli was accelerated when an electric field with a frequency in the range from 1 kHz to 1 MHz was applied. The degree of the acceleration of the growth of E. coli was up to about 130%, compared to when an electric field was not applied (control) or when an electric field with a frequency outside the above range was applied. The degree of acceleration may vary depending on temperature, time and the concentration of E. coli.

The present invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A method for accelerating the growth of E. coli comprising applying an A.C. electric field of 1 V to 10 MV/m with a frequency in the range from 1 kHz to 1 MHz or a D.C. electric field of comparable magnitude.

2. The method for accelerating the growth of E. coli according to claim 1, wherein the method is performed below 37° C.

3. The method for accelerating the growth of E. coli according to claim 1, further comprising collecting proteins from the culture medium of E. coli and isolating the same.

4. An apparatus for accelerating the growth of E. coli comprising:
a container (1) made of an insulating material;
two electrodes (2) equipped at the bottom of the container and the outer surface of a lid thereof; and
an electric field applying device (3) connected to the two electrodes,
wherein the electric field applying device applies an A.C. electric field of 1 V to 10 MV/m with a frequency in the range from 1 kHz to 1 MHz or a D.C. electric field of comparable magnitude.

5. The apparatus for accelerating the growth of E. coli according to claim 4, wherein one of the two electrodes is grounded and the other is connected to the anode or cathode of the electric field applying device.

6. The apparatus for accelerating the growth of E. coli according to claim 4, wherein the container contains E. coli and a culture medium.

7. The apparatus for accelerating the growth of E. coli according to claim 6, wherein the culture medium includes proteins excreted from E. coli.
